(19) United States
(12) Patent Application Publication
(10) Pub. No.: US 2025/0107932 A1
(43) Pub. Date: Apr. 3, 2025

(54) PROTECTIVE ASSEMBLY

(71) Applicant: 3M INNOVATIVE PROPERTIES COMPANY, St. Paul, MN (US)

(72) Inventors: Laurent Froissard, Cottage Grove, MN (US); Matthew B. Johnson, Woodbury, MN (US)

(21) Appl. No.: 18/891,072

(22) Filed: Sep. 20, 2024

Related U.S. Application Data
(60) Provisional application No. 63/541,972, filed on Oct. 2, 2023.

(51) Int. Cl. A61F 9/02

(52) U.S. Cl. CPC A61F 9/022; A61F 9/029

(57) ABSTRACT

A protective assembly including a singly-curved optically clear substrate and a laser-protective stack adhesively laminated to the substrate. The laser-protective stack includes a multilayer optical film configured to exhibit a light-transmission of 10% or less in a preselected wavelength range of the visible spectrum; the laser-protective stack will nevertheless exhibit an overall transmission of at least 60% over the visible spectrum. The laser-protective stack may also include an optically clear pressure-sensitive adhesive that adhesively bonds the multilayer optical film to the substrate, and may also include an optically clear physical-protection layer in front of the multilayer optical film.

### CROSS REFERENCE TO RELATED APPLICATIONS

This application claims priority from U.S. Provisional Application Ser. No. 63/541,972,filed 2 Oct. 2023, the disclosure of which is incorporated by reference in its/their entirety herein

### BACKGROUND

Many activities may benefit from vision protection from potential exposure to, e.g., laser radiation.

### SUMMARY

In broad summary, herein is disclosed a protective assembly including a singly-curved optically clear substrate and a laser-protective stack adhesively laminated to the substrate. The laser-protective stack comprises a multilayer optical film configured to exhibit a transmission of 10% or less in a preselected wavelength range of the visible spectrum; the laser-protective stack will nevertheless exhibit an overall transmission of at least 60% over the visible spectrum. The protective assembly may also comprise an optically clear pressure-sensitive adhesive that adhesively bonds the multilayer optical film to the substrate. The laser-protective stack may also comprise an optically clear physical-protection layer in front of the multilayer optical film. These and other aspects will be apparent from the detailed description below. In no event, however, should this broad summary be construed to limit the claimable subject matter, whether such subject matter is presented in claims in the application as initially filed or in claims that are amended or otherwise presented in prosecution.

### BRIEF DESCRIPTION OF THE DRAWINGS

FIGS. 1a and 1b are perspective views of exemplary personal-protection apparatus with which the herein-disclosed arrangements may be used.

FIG. 2 is a perspective view of another exemplary personal-protection apparatus with which the herein-disclosed arrangements may be used.

FIG. 3 is a perspective view of another exemplary personal-protection apparatus with which the herein-disclosed arrangements may be used.

FIG. 4 is a perspective view of an exemplary protective assembly comprising a substrate with a laser-protective stack thereon.

FIG. 5 is a top view of the exemplary protective assembly of FIG. 4.

FIG. 6 is a top view of another exemplary protective assembly.

FIG. 7 is a magnified view of a portion (indicated by the dashed-line circle in FIG. 5) of the exemplary protective assembly of FIG. 5.

FIG. 8 depicts a transmission spectrum of a hypothetical multilayer optical film.

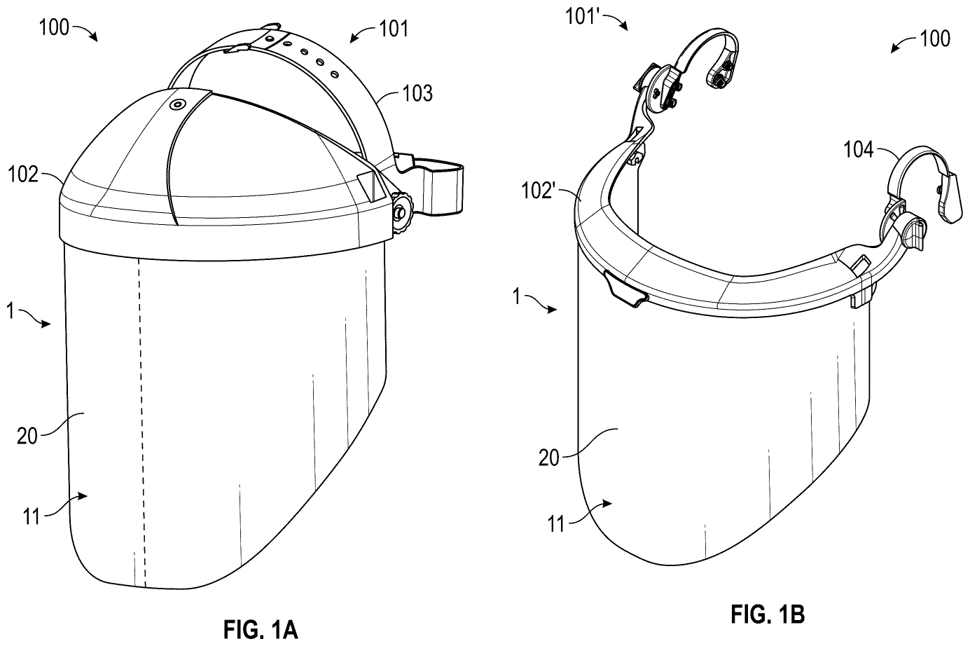

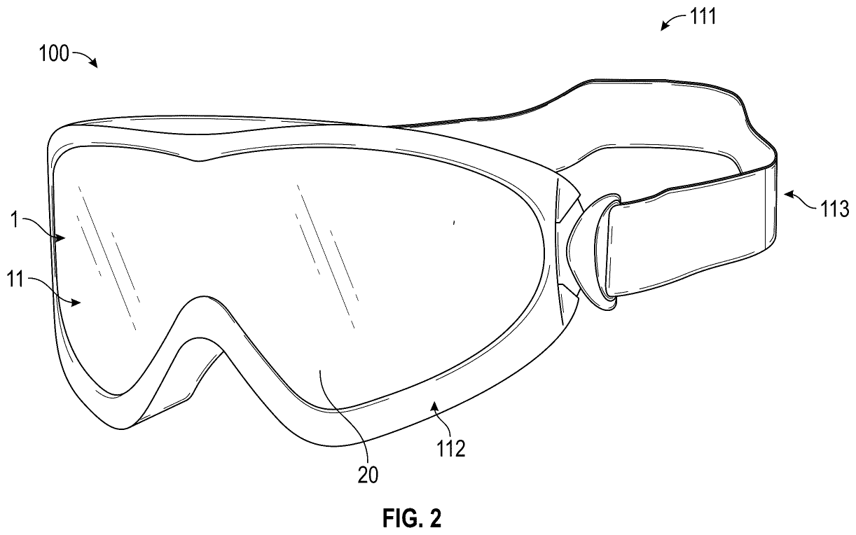

Like reference numbers in the various figures indicate like elements. Some elements may be present in identical or equivalent multiples; in such cases only one or more representative elements may be designated by a reference number but it will be understood that such reference numbers apply to all such identical elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated. Although terms such as “top”, bottom ”, “upper”, lower ”, “under”, “over”, “front”, “back”, “up” and “down”, and “first” and “second” may be used in this disclosure, it should be understood that those terms are used in their relative sense only unless otherwise noted. Terms such as outward, outwardmost etc., refer to a direction that, when a personal-protection apparatus is fitted to a user, is toward a source of laser radiation from which the user's eyes are desired to be shielded. Thus, for example, FIGS. 1 and 2 are views of the outward side of their respective persona-protection apparatus. Terms such as inward, inwardmost, etc., refer to a direction that is generally away from the outward direction (i.e., a direction that is toward the user's eyes).

### DETAILED DESCRIPTION

The term laser radiation is used in accordance with the customary meaning of the term; i.e., electromagnetic radiation generated by stimulated emission and that is spatially and temporally coherent.

By a laser-protective stack is meant an assembly of layers that collectively block at least one particular visible-light frequency range at which laser radiation may be present, the layers being non-separably joined together as a sheetlike assembly that can be handled as a single unit, so that the assembly can be laminated onto a visible-light-transmissive substrate (e.g., a pane) of a personal-protection apparatus.

By visible light, the visible spectrum, and like terminology, is meant electromagnetic radiation in the wavelength range of 380-680 nm. (These numbers are nominal values in view of the fact that the human eye does not have a sharp cutoff at these wavelengths but rather decreases smoothly as these wavelengths are approached and passed.)

By light-transmission is meant transmission of electromagnetic radiation (e.g. ultraviolet light, visible light, or infrared light) through a sheet-like material or set of materials (such as a multilayer optical film, an assembly comprising such a multilayer optical film, etc.), e.g. as measured with a spectrophotometer or by measuring the transmittance of a specific light source through the material. Such transmission may be measured in a particular range of visible light (ultraviolet or infrared); or it may be an “overall” light-transmission measured over the entire visible spectrum, in which case the transmission will generally correspond to “visible luminous transmission” as measured e.g. by ANSI Z87.1.

By a multilayer optical film is meant a film made of a plurality of coextruded, alternating, non-separable layers of a first organic polymer and a second, different organic polymer, which is designed to preferentially reflect radiation in at least one visible-light reflection band that is located in a predetermined wavelength range of the visible spectrum. In some embodiments such a film may exhibit one or more additional visible-light reflection bands that are located in one or more different predetermined wavelength ranges of the visible spectrum. In some embodiments such a film may broadly reflect or absorb non-visible light, e.g. ultraviolet (UV) radiation or infrared (IR) radiation.

By a singly curved substrate is meant a substrate that exhibits curvature only along a first axis and that is at least substantially flat along a second axis that is perpendicular to the first axis, as discussed in detail herein.

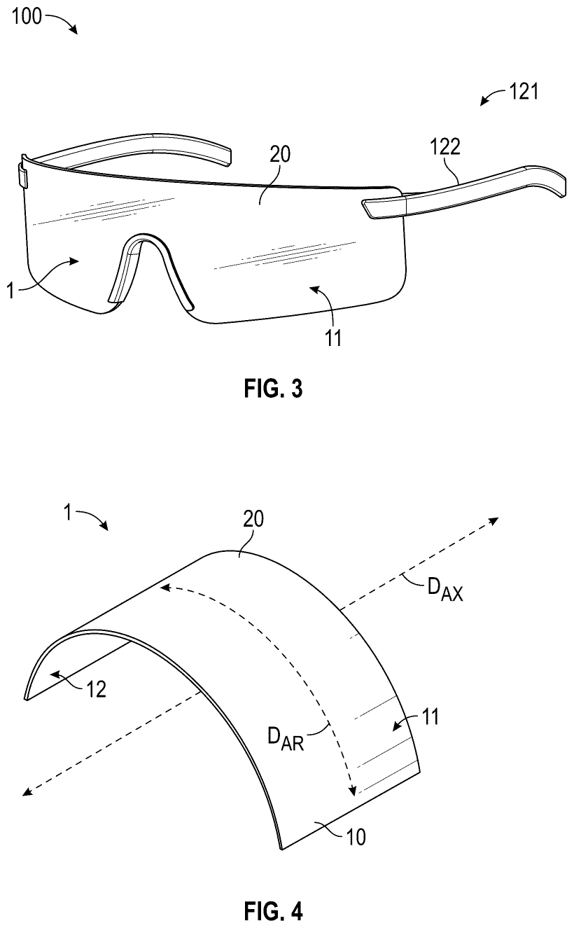

Disclosed herein is a protective assembly 1 that can be used e.g. to enhance the functioning of a personal-protective apparatus (e.g. protective eyewear, headgear, etc.). In some embodiments, such a personal-protection apparatus 100 may take the form of a visor 101 or 101′ as depicted in exemplary embodiment in FIGS. 1a and 1b. Visors 101 and 101′ as depicted are identical except for the arrangement of their frames 102 and 102′, with visor 101 comprising a head suspension 103 and visor 101′ comprising separate attachment elements 104 that may be used to, e.g. attach visor 101′ to a helmet or other headgear. In some embodiments, such a personal-protection apparatus 100 may take the form of goggles 111, comprising e.g. a frame 112 and a headband 113, as depicted in exemplary embodiment in FIG. 3. In some embodiments such a personal-protection apparatus 100 may take the form of eyeglasses 121 e.g. comprising arms 122 as depicted in exemplary embodiment in FIG. 2. Some such personal-protection apparatus 100 are configured to provide impact-protection against projectiles, propelled objects and the like, and may thus be configured with optically clear panes that are impact resistant.

Protection against laser radiation is becoming increasingly important. The arrangements disclosed herein allow a protective assembly 1 to be produced and incorporated into an impact-resistant personal-protection apparatus 100 such as a visor, helmet, goggles, eyeglasses, and the like, so that the protective assembly adds or augments protection against laser radiation.

Protective assembly 1 comprises a laser-protective stack 20, with the term “stack” denoting a set of sheet-like films, layers, etc. that are bonded together so as to not be separable from each other, stack 20 being bondable to a substrate 10 (e.g., to a pane of optically-clear, impact-resistant material) to form the protective assembly.

Laser-protective stack 20 will comprise at least one multilayer optical film 40. A multilayer optical film can be configured to provide very low transmission of visible light in at least one preselected wavelength range within the visible spectrum. In other words, a multilayer optical film can be configured to exhibit a relatively narrow-wavelength reflection band (sometimes referred to as a rejection band) in which the multilayer optical film strongly reflects incoming visible light, even as the multilayer optical film allows visible light in most or all other visible-light wavelength ranges to pass through substantially unattenuated. A multilayer optical film is thus ideally suited to be disposed e.g. on eyewear to protect the wearer from laser radiation while nevertheless allowing excellent visibility for the wearer.

A multilayer optical film, being comprised of numerous (e.g. hundreds) alternating layers, can be susceptible to warping, delamination, and so on, in the event that the multilayer optical film is exposed to differential stress (for example, if the multilayer optical film is bent into a multiply-curved shape). Multilayer optical films are also susceptible to such occurrences upon exposure to wide temperature swings, e.g. due to the different layers having slightly different thermal expansion coefficients, due to the layers comprising residual internal stresses (resulting from the stretching that such layers often undergo in the forming of the multilayer optical film), and so on. In some instances, such occurrences may result in e.g. delamination as manifested by gross separation of layers of the multilayer optical film; in other instances, such conditions may not result in gross separation but may nevertheless degrade the optical performance of the multilayer optical film.

The present work has revealed that a laser-protective stack 20 comprising a multilayer optical film 40 can be successfully disposed on a substrate 10 while minimizing or avoiding the above problems, if two conditions are met. First, the substrate upon which the laser-protective stack is disposed, should be a singly-curved substrate as described in detail herein. Second, the laser-protective stack should be bonded to the singly-curved substrate by being laminated to the substrate by way of a layer of pressure-sensitive adhesive. The first condition can provide that the laser-protective stack (in particular, its multilayer optical film) is not subjected to asymmetrical stretching during (and after) the act of bonding the stack to the substrate; the second condition can provide that the bonding can be accomplished without exposing the multilayer optical film to a temperature that is high enough to result in differential thermal stress, delamination, and so on.

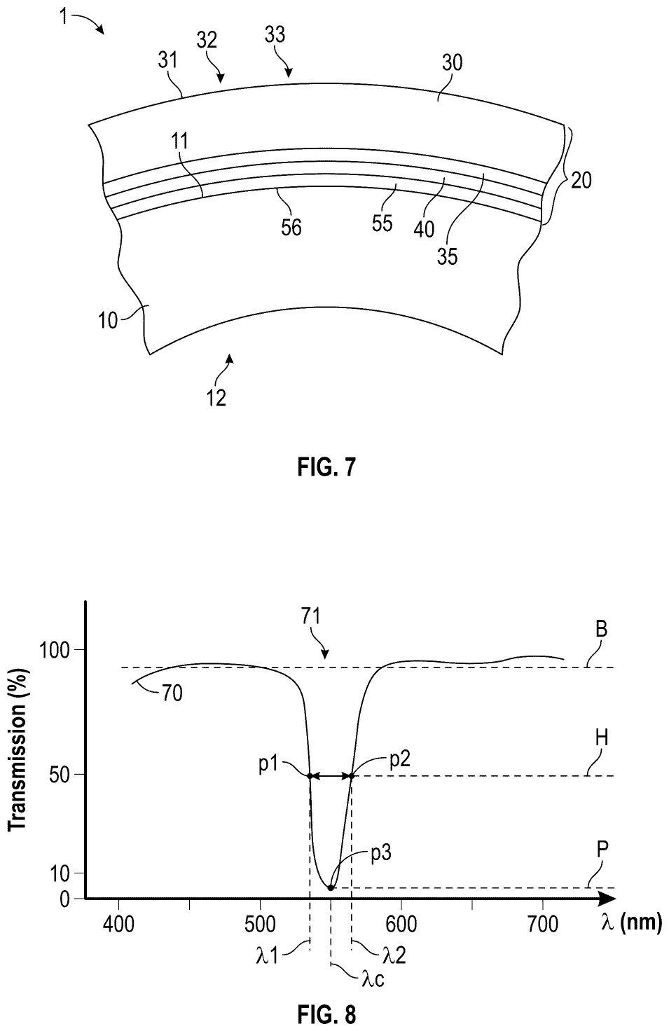

Thus in some embodiments, a laser-protective stack 20 as disclosed herein may comprise at least one pressure-sensitive adhesive (a “PSA”, e.g., an optically clear PSA) layer 55 that can be used to adhesively bond the rear side of the laser-protective stack 20 to a substrate. In some embodiments, a laser-protective stack 20 may comprise an optically clear physical-protection layer 30 that is positioned in front of the multilayer optical film 40, and may comprise a second optically clear PSA layer 35 that adhesively bonds a rear side of the optically clear protective layer to the front side of the multilayer optical film 40. (Such arrangements are visible in FIG. 7.) In some embodiments, other layers, treatments, and so on, may be present, as discussed herein.

Protective assembly 1 may be attached e.g. to a front side of a substrate 10 that is, or will become, a light-transmissive pane or lens of a personal-protection apparatus such as a visor or goggles. Here and elsewhere herein, the terminology of an entity being attached, bonded, laminated, etc., to a front (or rear) side of an item, encompasses the entity being affixed directly to a front (or rear) surface of the item, and also encompasses the entity being affixed to some other item (e.g. a layer, coating, etc.) that is disposed on the front (or rear) surface of the item.

Substrate 10 may be configured to protect the eyes of a user of the personal-protection apparatus, e.g. from projectiles, propelled objects, particulate debris, splashing liquids, and so forth. Substrate 10 may comprise any suitable material that meets the standards appropriate for a particular person-protection apparatus. In many embodiments substrate 10 may be an optically clear substrate e.g. of from 1 to 4 mm in thickness. In some embodiments such a substrate will be impact-resistant so as to meet the requirements of ANSI Z87.1-2020 (in particular, those requirements concerning a High Mass Impact Test and a High Velocity Impact Test) as well as Ballistic impacts as described in MIL-PRF-32432A or STANAG 4296 or STANAG 4495. In some embodiments, such a substrate will pass the Ballistic Impact Testing criteria provided in the Examples section of U.S. Patent Application Publication 2022/0410511, which is incorporated by reference in its entirety herein.

In many embodiments, substrate 10 will be optically clear. Here and elsewhere, by optically clear is meant that a layer, item, entity, etc., exhibits an overall light-transmission of at least 78% over the visible spectrum. In various embodiments, such a layer, item, entity, etc., will exhibit an overall light-transmission of at least 80, 85, or 90% over the visible spectrum. Thus in some embodiments, substrate 10 may comprise, or consist of, an optically clear organic polymeric material, e.g. a polycarbonate or a cyclic olefin polymer (e.g., a product available under the trade designation ZEONEX), and similar materials. In some embodiments, substrate 10 may be a multilayer structure. In some embodiments substrate 10 may comprise an organic polymeric layer with a scratch-resistant layer on a major surface thereof. This being the case, the concept of attaching a laser-protective stack 20 to a substrate 10 to form a protective assembly 1, encompasses the attaching of stack 20 to any kind of outermost layer (e.g., a scratch-resistant layer, hardcoat layer, an anti-fog coating, or the like) that may be present on substrate 10. In this regard, it is noted that a scratch-resistant or otherwise protective layer may be provided on a surface of substrate 10 in order to protect the surface during handling and assembly of substrate 10 into a protective assembly 1, even if the surface of the substrate will not be exposed during use of the protective assembly.

### Singly Curved Substrate

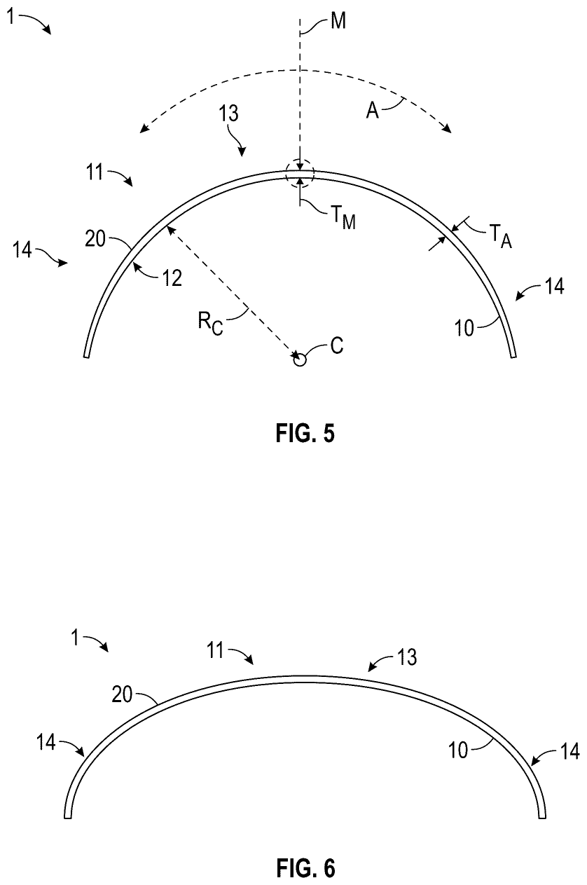

As mentioned above, substrate 10 will be singly curved in order to be able to laminate laser-protective stack 20 to a substrate 10 with minimum deformation, stresses, etc. By a singly curved substrate is meant a substrate that exhibits significant, readily discernable curvature along a first direction and is substantially flat along a second direction that is perpendicular to the first direction. (By substantially flat is meant having a radius of curvature of at least 2000 mm.) FIGS. 4 and 5 depict, in perspective view and in top view, an exemplary protective assembly comprising a singly curved substrate 10 (the protective assembly is likewise singly curved). In the depicted embodiment, singly curved substrate 10 is semi-cylindrical. Substrate 10 comprises a first, arcuate direction D_{AR} along which substrate 10 exhibits significant curvature, as evident in FIG. 4. Substrate 10 comprises a second direction D_{AX} (referred to herein as an axial direction) that is perpendicular to the first direction D_{AR} and along which substrate 10 is at substantially flat. Second, axial direction D_{AX} is parallel to the axis of symmetry of the right circular cylindrical sectional shape exhibited by substrate 10.

In various embodiments, substrate 10 and the protective assembly that includes substrate 10, may exhibit a radius of curvature R_{C} (pointed out in FIG. 5, along with the approximate center of curvature C) of from 50 to 300 mm. In various particular embodiments, the radius of curvature may be e.g. approximately 88, 66, or 50 mm. In various embodiments, substrate 10 and the protective assembly that includes substrate 10, may exhibit a radius of curvature in the second direction that is at least 20000 mm, or is e.g. essentially infinite.

Substrate 10 is thus singly curved. In further detail, singly curved substrate 10 will exhibit a so-called developable surface that will exhibit a Gaussian curvature of substantially zero. Intuitively, a singly curved, developable surface is one that can be mentally “unrolled” onto a planar surface without distortion. A singly curved, developable surface can be contrasted to one that is multiply (e.g. doubly) curved. A multiply curved surface will exhibit more than one axis along which the surface exhibits significant curvature. Examples of multiply curved surfaces are spheres, which exhibit non-zero, positive Gaussian curvature, and many hyperbolic surfaces, which exhibit non-zero, negative Gaussian curvature. A singly curved surface can be contrasted with surfaces of prescription eyeglasses, which, being typically formed from spherical lens blanks and having special shapes formed into their rear surfaces to provide the desired optical power, are typically multiply curved. A singly-curved substrate 10 as disclosed herein is well suited to allow a laser-protective stack 20 (which may be obtained in the form of a continuous roll that is singly curved) to be laminated thereto with minimum difficulty.

Various geometric parameters of singly-curved substrate 10 can be considered with reference to FIG. 5, which is a top view of protective assembly 1 and its substrate 10, looking along the above-described second, axial direction DAX. In some embodiments, substrate 10 may take the shape of a uniform right circular cylinder (as with the exemplary assembly and substrate depicted in FIG. 5). In such a case, the radius of curvature R_{C} may not vary at different locations of substrate 10 but may rather be constant along the entirety of an angular arc that extends along the arcuate direction DAR of the substrate/assembly from the arcuate midline (M) of the substrate to the left and right terminal edges of the substrate. (By constant is meant within plus or minus 5%; in various embodiments the radius of curvature may vary less than plus or minus 3%, 1.0%, or 0.5%, along the entirety of arcuate direction of the substrate.)

However, in some embodiments, lateral edge sections 14 that are proximate the left and right terminal edges of substrate 10, may exhibit a somewhat different radius of curvature than that in a midsection 13 of substrate 10. In other words, in some instances substrate 10 (and the assembly 1 which it is a part of) may have lateral edge sections 14 that are slightly flared outward, slightly curled inward, and so on. For example, the exemplary substrate 10 (and the protective assembly 1 of which the substrate is a component) depicted in FIG. 6 exhibits lateral edge sections 14 that are somewhat curled inward e.g. to achieve a “wraparound” effect.

However, in many embodiments (e.g. including those in which the radius of curvature in lateral edge sections 14 differs from that in midsection 13), substrate 10 and assembly 1 may exhibit a uniform radius of curvature at least in a midsection 13 of substrate 10. Thus in some embodiments, substrate 10 may exhibit a shape that, when viewed along the axial direction DAX of the substrate (as in FIGS. 5 and 6), exhibits an at least substantially constant radius of curvature Rc at all locations of the substrate that lie on an arc (an exemplary arc A is depicted in FIG. 4) that is centered on the midline M of the substrate and that extends e.g. 20, 40, or 60 degrees to each side of midline M along the arcuate direction DAR of substrate 10.

### Variable-Thickness Substrate

In some embodiments, substrate 10 can exhibit a thickness that is uniform throughout the length and breadth of substrate 10. In many embodiments, substrate 10 may be uniform in thickness along the axial direction D_{AX}. However, in some embodiments substrate 10 may exhibit a thickness that varies along the arcuate direction D_{AR}. In some particular embodiments, substrate 10 may exhibit a maximum local thickness at a midline of the substrate (as denoted by thickness T_{M} in FIG. 5) with the local thickness of the substrate decreasing as a path is followed along the arcuate direction D_{AR} of substrate 10 away from the midline M of the substrate. In many embodiments the thickness may smoothly decrease as the distance from the midline increases as in the exemplary depiction of FIG. 5. The rate at which the thickness decreases may be suitably chosen, e.g. in order to avoid any changes in the refractive power exhibited by the substrate (and the resulting protective assembly) along the arcuate direction of the substrate/assembly.

Such arrangements may be chosen e.g. according to the Gullstrand Formula, as described in U.S. Patent Application Publication 2006/0098161, which is incorporated by reference in its entirety herein for this purpose. Such arrangements may alternatively be chosen e.g. according to the well-known optical formula for the inner radius of a focal lens, which relates the inner radius, outer radius, and thickness of a lens or pane, as a function of the refractive index of the lens material and according to the desired optical power. In the present case, it will typically be advantageous that the optical power be close to zero along the arcuate direction (as well as along the axial direction) e.g. to avoid blurring or distortion near the arcuate edges of the protective assembly.

By way of a particular example, a substrate (e.g. with an outer radius of 106 mm) may have a local thickness T_{M} at the midline M of the substrate of e.g. 2.1 mm, with the thickness decreasing so that so that a local thickness of the substrate at a location that is approximately 60 degrees away from the midline M (e.g., thickness T_{A} as denoted in FIG. 5) is approximately 1.5mm. Thus in various embodiments, the local thickness of substrate 10 at a location that is 60degrees away from the midline M, may be from e.g. 50 to 60, up to 80 or 90, % of the local thickness of the substrate at the midline. Such arrangements can provide that the optical power (refraction) exhibited by substrate 10 (and thus exhibited by a protective assembly resulting from the lamination of a laser-protective stack 20 to substrate 10) in its left and right lateral edge sections 14 will not vary significantly from its value near the substrate midline.

### Multilayer Optical Film

Laser-protective stack 20, as laminated to a substrate 10 to form a protective assembly 1, will comprise at least one multilayer optical film 40 as depicted in exemplary embodiment in FIG. 7 (the individual film layers within multilayer optical film 40 are not shown). By a multilayer optical film is meant a plurality (often, hundreds) of coextruded, alternating layers of a first organic polymer and a second, different organic polymer, the layers being designed to preferentially reflect radiation in at least one visible-light reflection band that is located in a predetermined wavelength range of the visible spectrum. In some embodiments such a film may exhibit one or more additional visible-light reflection bands that are located in one or more different predetermined wavelength ranges of the visible spectrum. In some embodiments such a film may broadly reflect non-visible light, e.g. ultraviolet (UV) radiation or infrared (IR) radiation, as discussed in detail later.

The ordinary artisan will understand that a multilayer optical film generally operates as a quarter-wave stack, in which constructive and destructive interference from the alternating, optically active layers collectively provide a reflection spectrum. The thickness of the individual layers, the total number of layers, the refractive indices of the layers, and so on, may be chosen in combination to provide a predetermined reflection spectrum. In particular, by appropriate selection of materials, processing conditions, and thicknesses of the layers, the transmission spectrum can be tailored to provide a strong reflection band (hence can provide strong blocking of visible light) in a narrow band. A multilayer optical film may thus be particularly well suited for providing protection from laser radiation emitted from portable lasers, since portable lasers often rely on light-emitting diodes that emit laser radiation at a particular, fixed wavelength.

In various embodiments, an multilayer optical film (and thus, a laser-protective stack that includes such a multilayer optical film, and a protective assembly that comprises the laser-protective stack) can block a narrow band of visible light in a predetermined wavelength range that corresponds to, e.g.: green laser radiation (which is often centered near 520 or 532 nm); red laser radiation (which is often centered near 630-670 nm); orange laser radiation (which is often centered near 594 nm); blue laser radiation (which is often centered around 445 nm); and/or violet laser radiation (which is often centered around 405 nm). In many embodiments, the multilayer optical film can achieve an optical density of at least 4, 5 or 6 in the predetermined wavelength range(s).

Such behavior can be assessed with reference to FIG. 8, which presents, in idealized representation, a visible-light transmission spectrum for a hypothetical multilayer optical film. In FIG. 8, light-transmission (as a percent) is plotted against optical wavelength λ as curve 70. Curve 70 can be considered to represent the transmission of light through the multilayer optical film at normal incidence. (The light-transmission will vary somewhat as a function of incidence angle; all numbers herein will be understood to be obtained under nominal conditions corresponding to normal incidence, or as near to normal incidence as is possible for any particular measuring apparatus.) The hypothetical multilayer optical film of FIG. 8 is seen to selectively block light within a narrow light-rejection band in the green region of the visible spectrum, evidenced by the presence of low-transmission notch 71 in curve 70.

In order to quantify relevant features of curve 70, the following parameters are of interest: an average, best-fit visible-light-transmission baseline B of curve 70, a peak value P of curve 70 (noting that the peak value P corresponds to a transmission minimum, at point p3), and an intermediate value H of curve 70, halfway between P and B. Curve 70 intersects with the value H at the points p1 and p2, whose wavelength values equal the short wavelength band edge λ1 and the long wavelength band edge 22, respectively, of the rejection band. The short and long wavelength band edges can be used to calculate two other parameters of interest: the width (full width at half-maximum, or FWHM) of the light-rejection band, which equals λ2-λ1; and the center wavelength Ac of the rejection band, which equals (λ1+λ2)/2. The center wavelength Ac may be the same as or different from the peak wavelength (see point p3) of the rejection band, depending on how symmetrical the rejection band is.

Light-transmission (in general, the transmission of electromagnetic radiation, including e.g. infrared light and/or ultraviolet light), refers generally to the transmitted intensity divided by the incident light intensity, and can be evaluated e.g. as a function of wavelength using a spectrophotometer using well-known methods (e.g., the methods and apparatus described in U.S. Pat. No. 10,948,745). For an optical element (e.g. a multilayer optical film, a laser-protective stack comprising a multilayer optical film, a protective assembly comprising a substrate along with a laser-protective stack, and so on), the percent transmission in any particular wavelength range can be straightforwardly obtained by such methods. In some embodiments, the light transmission in a wavelength range may be characterized in terms of optical density (negative of the base 10 logarithm of the light-transmission).

For a substrate and/or protective assembly, an overall visible-light transmission (i.e., an average light-transmission over the entire visible spectrum) can be similarly obtained (and will generally be proportional to the area under curve 70 in the visible light spectrum). The procedure for obtaining overall visible-light transmission may generally correspond to the procedures outlined in the Transmittance and Luminous Transmittance Tests found in ANSI Z87.1-2020 and the resulting overall light-transmission value may closely approximate the parameter known in the art as visible luminous transmittance.

Testing of light-transmission (whether in particular wavelength ranges, obtaining an overall value of visible luminous transmittance, etc.), optical density, and so on, may also be performed via laser-based measurement systems such as those available from Spica Technologies (Hollis, New Hampshire). In some embodiments, a substrate 10 and/or a protective assembly I may be tested in general accordance with the apparatus and procedures found in ANSI Z136.7-2020 (Testing and Labeling of Laser Protective Equipment) and/or in EN ISO 19818-1:2021 (Eye and face protection-Protection against laser radiation).

Because a multilayer optical film can be configured to block light only in a very narrow band, allowing the remainder of the visible light to pass through, the multilayer optical film can allow excellent overall light transmission and thus can allow excellent visibility through a protective assembly of which the multilayer optical film is a part. In various embodiments, a multilayer optical film, and/or a protective assembly comprising such a film, may exhibit an overall light-transmission, over the visible-light spectrum, of at least 60, 70, 80, 90, or 95%. In various embodiments, any such film or assembly may exhibit a light-transmission of at least 60, 70, 80, or 90% at every individual visible-light wavelength that is not located within the reflection band or bands. (In other words, a multilayer optical film can be configured so that the visible light transmission is very high everywhere except in a reflection band.)

In various embodiments, a multilayer optical film, and/or a protective assembly comprising such a film, may exhibit at least one reflection band in a preselected wavelength range of the visible spectrum, in which range the light-transmission spectrum exhibits a peak (P) (i.e., a transmission minimum) at which wavelength the light-transmission is less than 10, 4, 2, or 1%. In some embodiments, the multilayer optical film and/or the protective assembly may exhibit, at such a peak of a preselected wavelength range, an optical density (measured e.g. using a transmission densitometer) of a least 4, 5, or 6. In some embodiments, a reflection band may exhibit a width (i.e., a full width at half-maximum, or FWHM as discussed above) of less than 100, 80, 60, 40 20, or 10 nm.

In various embodiments, such a reflection band and its preselected wavelength range may correspond to the above-cited wavelength ranges of green laser light, orange laser light, blue laser light, and/or violet laser light. In some embodiments, only a single visible-light reflection band will be present (e.g. configured to block green laser radiation). In other embodiments, the multilayer optical film may be configured to exhibit multiple (e.g., two) visible-light reflection bands (e.g. configured to block green and red laser radiation). In some embodiments, a laser-protective stack may comprise two separate multilayer optical films (e.g. separated by a layer of optically clear pressure-sensitive adhesive), one such film configured to block one preselected wavelength (e.g. green) and the other configured to block a different preselected wavelength (e.g. red).

One or more multilayer optical films 40, each having alternating layers of at least one polymer (e.g. a birefringent polymer) and one second polymer, may be used in protective assembly 1. Materials suitable for making an at least one birefringent layer of a multilayer optical film include organic polymers (e.g., polyesters, copolyesters, and modified copolyesters). In this context, the term “polymer” will be understood to include homopolymers and copolymers, as well as polymers or copolymers that may be formed in a miscible blend, for example, by co-extrusion or by reaction, including transesterification. The terms “polymer” and “copolymer” include both random and block copolymers. Polyesters suitable for use in some exemplary multilayer optical films constructed according to the present disclosure generally include carboxylate and glycol subunits and can be generated by reactions of carboxylate monomer molecules with glycol monomer molecules.

An exemplary polymer useful as the birefringent layer in multilayer optical films is polyethylene naphthalate (PEN), which can be made, for example, by reaction of naphthalene dicarboxylic acid with ethylene glycol. Polyethylene 2,6-naphthalate (PEN) is frequently chosen as a birefringent polymer. PEN has a large positive stress optical coefficient, retains birefringence effectively after stretching, and has little or no absorbance within the visible range. PEN also has a large index of refraction in the isotropic state. Its refractive index for polarized incident light of 550 nm wavelength increases when the plane of polarization is parallel to the stretch direction from about 1.64 to as high as about 1.9. Increasing molecular orientation increases the birefringence of PEN. The molecular orientation may be increased by stretching the material to greater stretch ratios and holding other stretching conditions fixed. Copolymers of PEN (CoPEN), such as those described in U.S. Pat. Nos. 6,352,761 and 6,449,093 are particularly useful for their low temperature processing capability making them more coextrusion compatible with less thermally stable second polymers. Other semicrystalline polyesters suitable as birefringent polymers include, for example, polybutylene 2,6-naphthalate (PBN), polyethylene terephthalate (PET), and copolymers thereof such as those described in U.S. Pat. No. 6,449,093 and U.S. Patent Application Publication 20060084780. Alternatively, syndiotactic polystyrene (sPS) is another useful birefringent polymer.

The second polymer of a multilayer optical film can be made from a variety of polymers e.g. having glass transition temperatures compatible with that of the first birefringent polymer and having a refractive index similar to the isotropic refractive index of the birefringent polymer. Examples of other polymers suitable for use in optical films and, particularly, in the second polymer include vinyl polymers and copolymers made from monomers such as vinyl naphthalenes, styrene, maleic anhydride, acrylates, and methacrylates. Examples of such polymers include polyacrylates, polymethacrylates, such as poly (methyl methacrylate) (PMMA), and isotactic or syndiotactic polystyrene. Other polymers include condensation polymers such as polysulfones, polyamides, polyurethanes, polyamic acids, and polyimides. In addition, the second polymer can be formed from homopolymers and copolymers of polyesters, polycarbonates, fluoropolymers, and polydimethylsiloxanes, and blends thereof.

Other exemplary suitable polymers, especially for use as the second polymer, include homopolymers of polymethylmethacrylate (PMMA), such as those available from Ineos Acrylics, Inc., Wilmington, DE, under the trade designations CP71 and CP80, or polyethyl methacrylate (PEMA), which has a lower glass transition temperature than PMMA. Additional second polymers include copolymers of PMMA (coPMMA), such as a coPMMA made from 75 wt % methylmethacrylate (MMA) monomers and 25 wt % ethyl acrylate (EA) monomers, (available from Ineos Acrylics, Inc., under the trade designation Perspex CP63), a coPMMA formed with MMA comonomer units and n-butyl methacrylate (nBMA) comonomer units, or a blend of PMMA and poly(vinylidene fluoride) (PVDF).

In general, the selection of the polymer compositions used in creating the multilayer optical film can be made in view of the desired properties (e.g., centerpoint, width, band edge sharpness, etc.) of the visible-reflection band or bands. Higher refractive index differences between the birefringent polymer and the second polymer create more optical power thus enabling more reflective bandwidth. Alternatively, additional layers may be employed to provide more optical power. Methods of increasing (steepening) the slope of a band edge are disclosed e.g. in U.S. Pat. No. 6,157,490, which is incorporated by reference herein in its entirety for this purpose. Multilayer optical films, materials suitable for use in such films, and/or methods of making such films, are described in detail e.g. in U.S. Pat. Nos. 6,783,349, 7,271,951, 8,441,724, 10,054,803, and 10,948,745; and, in U.S. Patent Application Publications 2009/0283144, 2016/0216427, and 2022/0410511; all of which are incorporated by reference in their entirety herein.

In some embodiments, two or more multilayer optical films (whether similar or identical to each other, or differing significantly e.g. in their number of layers and/or the composition of the layers) may be used in protective assembly 1. Thus in some embodiments, a laser-protective stack and a protective assembly that includes such a stack, may include two or more multilayer optical films, that are held together (whether directly or with one or more intervening layers) via an optically clear pressure sensitive adhesive).

In some embodiments it may be desirable for a laser-protective stack 20 to also be able to broadly block infrared radiation (meaning radiation with a wavelength over 680 nm) and/or ultraviolet radiation (meaning radiation with a wavelength under 380 nm). For example, green (e.g. 532 nm) laser radiation is often produced by first producing 1064 nm laser radiation and then performing frequency doubling. In such a case, significant amounts of 1064 nm laser radiation may be present in the “green” laser beam.

Thus in some embodiments, a multilayer optical film may be configured to block ultraviolet and/or infrared radiation in addition to exhibiting at least one reflection band in the visible range. In such a case, the light transmission may being to drop at or near the long-and-short-wavelength edges of the visible spectrum (the onset of such a drop in transmittance at the ultraviolet (short wavelength) edge of a spectrum may be seen at the left end of curve 70 of FIG. 8). In some embodiments, a first multilayer optical film may be present in order to block narrowband laser radiation at a preselected wavelength, with another, separate multilayer optical film being present in order to broadly block infrared radiation, and/or with another, separate multilayer film being present in order to broadly block ultraviolet radiation. Configurations of multilayer optical films that can provide blocking of infrared radiation and/or ultraviolet radiation are described e.g. in U.S. Pat. Nos. 7,271,951, 8,441,724, 10,444,546, 10,948,745 and 10,962,806, and in U.S. Patent Application Publication 2022/0410511, all of which are incorporated by reference in their entirety herein.

In some embodiments, laser-protective stack 20 may comprise at least one layer that is not a multilayer optical film and that is configured to block ultraviolet radiation and/or infrared radiation. In some embodiments, such a layer may rely on broad-band absorption via the inclusion of dyes, pigments, etc. in the layer. A non-limiting list of materials that may be suitable for use as an ultraviolet blocker includes hindered-amine light stabilizers (HALS), benzotriazoles, benzophenones, oxanilides, benzoxazinones, and like materials (such materials are often referred to as UV absorbers or UV stabilizers). A non-limiting list of materials that may be suitable for use as an IR blocker includes tin, antimony, indium, tin oxide, antimony oxide, indium oxide, indium-doped tin oxide, and mixtures thereof. In some embodiments at least one IR blocker and at least one UV blocker may be used in combination (whether in the same layer, or in different layers).

In various embodiments, a laser-protective stack 20 (and a protective assembly comprising such a stack) that is configured to provide IR and/or UV blocking (whether achieved by way of one or more multilayer optical films, one or more absorptive layers, or a combination thereof) may exhibit an optical density throughout the ultraviolet range (e.g., 100 nm to 380 nm) that is at least 2, 3, 4, 5 or 6. Similarly, a laser-protective stack 20 (and a protective assembly comprising such a stack) that includes any such arrangement may exhibit an optical density in at least the near-infrared range (e.g. 680 nm to 1300 nm), or throughout the entire infrared range (e.g. up to approximately 1 mm), that is at least 2, 3, 4, 5 or 6.

In some embodiments, a laser-protective stack 20 may comprise an optically clear physical-protection layer 30 that is disposed forward of the multilayer optical film 40 of the stack, as in the exemplary design shown in FIG. 7. By a physical-protection layer 30 is meant any layer that protects some other, underlying layer (e.g., a multilayer optical film) from mechanical damage, from abrasion from particulate debris, and so on. Any suitable organic polymeric material may be used for such a layer. In many embodiments, a layer of polycarbonate, cyclic olefin polymer, or similar material (and with a thickness of e.g. from 0.05 to 0.3 mm), may serve this purpose. In some embodiments, such a layer 30 may be adhesively bonded to the front side of multilayer optical film 40 (or to a layer thereon) by way of a layer of optically clear adhesive 35 as shown in FIG. 7.

Although materials such as e.g. polycarbonate can provide excellent physical protection e.g. for a multilayer optical film, the front surface 31 of such a physical-protection layer 30 may be susceptible to being scratched, scuffed, etc. So, in some embodiments, front surface 31 of physical-protection layer 30 may comprise a scratch-resistant layer (e.g. a coating) 32, sometimes referred to as a hardcoat. A non-limiting list of potentially suitable hardcoats includes acrylic hardcoats, silica-based hardcoats, siloxane hardcoats, melamine hardcoats, and the like (noting that there may be overlap of some of these categories). In some embodiments, such a hardcoat layer may be e.g. coated onto the physical-protection layer 30 and solidified/hardened by curing to form a robustly cross-linked layer. Various hardcoat compositions that may be suitable are described e.g. in U.S. Patent Application Publications 2020/0354607 and 2022/0410511, both of which are incorporated by reference herein in their entirety.

In some embodiments, the front surface 31 of the physical-protection layer 30 may comprise an anti-reflective layer 33 (which may be in the form of e.g. a coating, a surface treatment, etc.). In some embodiments a hardcoat layer may serve as both a scratch-resistant layer and as an anti-reflective layer. In many embodiments, physical-protection layer 30, or a scratch resistant and/or anti-reflective layer thereon, will comprise the forwardmost (and outermost) surface of the laser-protective stack 20. It is noted in passing that in some embodiments, a rearmost surface of the rear side 12 of substrate 10 may similarly comprise an anti-reflective layer, treatment, or coating, it being known that such treatments are most often needed at solid-air interfaces.

In some embodiments, the front surface 31 of the physical-protection layer 30 may comprise an anti-fog or anti-mist layer (which again may be in the form of e.g. a coating, surface treatment, etc.). Exemplary compositions that may be suitable for providing an anti-fog layer are disclosed e.g. in U.S. Patent Application Publication 2010/0092765.

A laser-protective stack 20 will comprise an optically clear pressure sensitive adhesive (PSA) layer 55 that serves to adhesively bond the rear side of the stack 20 (e.g., a rearmost side of a multilayer optical film 30 of stack 20) to a front side of substrate 10. Such an adhesive layer 55 will thus often comprise the rearmost layer of the laser-protective stack 20, with a rear major surface 56 of adhesive layer 55 being bonded to the front major surface 11 of substrate 10 (noting that the front major surface of substrate 10 may be the actual surface of substrate 10, or the front surface of any layer, coating, treatment, etc., present thereon). The present investigations have revealed that the most advantageous results are obtained when the adhesive layer 55 that bonds stack 20 to substrate 10, is greater than 5 microns in thickness. In various embodiments, adhesive layer 55 may be from 10, 15, 20, or 25, to 130, 100, 80, 50, 40, or 30microns in thickness. As noted above, in some embodiments stack 20 may comprise a physical-protection layer 30 that is disposed forward of the multilayer optical film 30, and may include a second optically clear PSA layer 35 that serves to adhesively bond the rear side of physical-protection layer 30 to the other layers of the stack. In some embodiments, optically clear PSA layer 35 will bond protection layer 30 to the front side of multilayer optical film 40. Such a PSA layer can be any suitable thickness, e.g. down to 5 microns in thickness.

The term pressure-sensitive adhesive is well understood to denote an adhesive that meets the well-known Dahlquist criterion described in the Handbook of Pressure-Sensitive Adhesive Technology, D. Satas, 2^{nd} ed., page 172 (1989). This criterion defines a pressure-sensitive adhesive as one having a one-second creep compliance of greater than 1×10⁻⁶ cm²/dyne at its use temperature (for example, at temperatures in a range of from 15° C. to 35° C.). Many optically clear PSAs are available, for example the products available from 3M Company under the trade designation OPTICALLY CLEAR ADHESIVE 8211, 8212, 9213, 8214, and 8215. A laser-protective stack 20, as initially constructed, may include a removable release liner e.g. on a first optically clear PSA layer 55 that is to be used to attach the stack to a front surface of a protective substrate; such a release liner will be removed prior to the attaching of the stack to the surface of the protective substrate.

A laser-protective stack 20 may be generated by laminating a physical protection layer 30 onto a multilayer optical film 40 e.g. via an optically clear PSA and laminating another optically clear PSA 55 to the rear of this structure to be used to laminate the laser-protective stack 20 to a substrate 10. If any additional layers are present in the laser-protective stack (e.g. an additional multilayer optical film, a UV-absorptive layer and/or an IR-absorptive layer, etc.), these may likewise be laminated by way of additional layers of optically clear PSAs.

A singly-curved optically clear substrate may be produced by any suitable method, e.g. by extrusion, profile extrusion, injection molding, compression molding, injection-compression molding, and so on. Such methods can be configured to produce a substrate that, as discussed in detail earlier herein, exhibits a desired radius of curvature at various locations along an arcuate direction of the substrate, a thickness that decreases smoothly from a midline of the substrate toward its arcuate edges, and so on.

The lamination to adhesively bond laser-protective stack 20 to substrate 10 can be done e.g. by passing substrate 10, and laser-protective stack 20, through a lamination nip between two nip rolls that are pressed together with a suitable force. The substrate 10 and the stack 20 will be oriented so that the optically clear PSA 55 of the laser-protective stack comes into contact with the front surface 11 of substrate 10 so as to adhesively bond thereto. The substrate will be passed through the nip in a direction that is aligned with the arcuate direction DAR of the substrate. Although the substrate, as made, comprises curvature along this arcuate direction as discussed in detail earlier herein, this will not prevent the substrate from being fed into the lamination nip. (As the substrate passes through the lamination nip, it may be at least momentarily, locally urged into a slightly less curved configuration, although it will tend to resume its “natural” curvature once the laminating pressure abates.)

Also as discussed earlier herein, in some embodiments substrate 10 may exhibit a variable thickness along the arcuate direction D_{AR} of the substrate. A lamination nip can be configured so that this will not prevent (or cause any problems with) the lamination, e.g. by providing that at least one of the nip rolls is covered with a compliant, resilient material, for example natural rubber, nitrile rubber, silicone rubber, or the like. Even if both of the nip rolls are hard-surfaced (e.g. if both nip rolls have steel surfaces), the rolls may be pressed together with a prechosen force, with the gap between the nip rolls being allowed to vary while maintaining a constant force, rather than this gap being set e.g. at a predetermined, unchangeable value. Such arrangements can provide that lamination can be successfully performed even with a substrate whose thickness varies along its arcuate length.

In some embodiments, a laser-protective stack can be laminated to a substrate that is in the form of a lens blank, with the lens blank and the laser-protective stack then being cut down to the proper size and shape to provide the final protective assembly (e.g. for use as a pane of a visor or of single-pane goggles, for use as an individual pane of double-pane goggles, and so on). In some embodiments a substrate may already have been cut and shaped into its nominal final form (e.g. for use as a pane of a visor or of single-pane goggles, etc.) when the laser-protective stack is laminated thereto. The laser-protective stack and/or the substrate may then be edge-trimmed as needed to provide the final protective assembly.

As mentioned, an advantage of the present arrangements is that the formation of laser-protective stack 20, and the attachment of stack 20 to a substrate 10, can be achieved via lamination that is done at relatively low temperature and that is done while imparting a minimum of stretching or distorting force to the multilayer optical film contained in the stack. Within this limits, a slightly elevated lamination temperature may ensure that the pressure-sensitive adhesive is able to wet out against each bonded layer extremely well. In various embodiments, such lamination may be done at a temperature (corresponding to a nominal set point temperature at which one or both of the nip rolls is set) that ranges e.g. from room temperature (e.g. 21 C), to a maximum of 90, 80, 60, or 40 F. It is noted that whatever the lamination conditions, the lamination should be performed so that in the finished assembly, there are no air gaps between any of the optically clear adhesive layers and any of the bonded layers (e.g. the multilayer optical film, the physical-protection layer, and the substrate). In many embodiments, the layers of laser-protective stack 20 will not be separable from each other; similarly, once stack 20 is laminated to substrate 10 to form protective assembly 1, stack 20 will not be removable from substrate 10 without damaging or destroying stack 20 and/or substrate 10.

A protective assembly 1 as disclosed herein can be incorporated into personal-protection apparatus such as a visor, helmet, goggles, and the like (in general, into any protective eyewear or headgear, as depicted in exemplary embodiments in FIGS. 1-3), so that the protective assembly provides protection against laser radiation. Typically, such a protective assembly will be incorporated into a personal-protective apparatus (e.g. will be fitted to a frame 102 or 102′ to form a visor, fitted to a frame 112 to form goggles, fitted to arms 122 to form glasses, etc.), in a shape that is very close to the “natural” or inherent curvature exhibited by substrate 10 in the absence of any deforming force. In some embodiments, the installation of the assembly into a frame such as for goggles or a visor, may impart a slight force on the substrate (e.g. so that the substrate is held firmly by the frame); however, this force will typically be rather small and will not cause problems for the protective assembly (in particular, will not cause e.g. delamination of the multilayer optical film).

In some embodiments, any such eyewear or headgear may comprise a single-piece protective assembly as with the exemplary arrangements depicted in FIGS. 1-3 (such eyewear is often referred to as having a single-pane lens). In some embodiments, a separate protective assembly may be provided for each eye of a user, with each protective assembly installed in a separate portion of a frame in the manner of conventional eyeglasses (such eyewear is often referred to as having dual-pane lenses). The two protective assemblies will not necessarily lie on a common curve, but each assembly will nevertheless be singly-curved as described herein. In generally all envisioned embodiments, any such protective assembly will be installed in a personal-protective apparatus (e.g., eyewear or headgear) so that the previously-described axial direction of the protective assembly is oriented generally vertically when the user is standing.

Discussions so far herein have concerned incorporating a laser-protective stack into a protective assembly whose substrate is configured to provide enhanced impact resistance. This may be particularly useful in the case of protective eyewear and/or headgear worn by armed forces, law enforcement, and the like. However, this may not be necessary in all embodiments. (For example, airline pilots and military pilots are typically shielded from impacts by the cockpit windshield; nevertheless, it may be desirable to protect the pilot from laser radiation.) This being the case, in some embodiments a laser-protective stack 20 may be incorporated into a protective assembly that is not configured to have enhanced impact resistance, e.g., that will not meet the above-mentioned Impact Resistance standards of ANSI Z87.1-2020 and/or the previously mentioned military standards (MIL and STANAG). (Such an assembly will be referred to in general as non-impact-resistant, although it may still provide some degree of protection from impacts.) For example, in some embodiments a non-impact resistant protective assembly 1 can be used (e.g. in the form of a flip-down visor, shield, etc.) as an adjunct to an impact-protective eyewear, headgear, helmet or the like. Or, in the above example of pilots, a non-impact-resistive protective assembly I might be provided as non-impact-resistive eyeglasses, goggles, a visor, or the like, that can be donned in the cockpit if needed.

Similarly, discussions so far herein have concerned incorporating a laser-protective stack into a protective assembly so as to provide narrow-band protection against laser radiation while nevertheless maintaining the highest possible overall visual transmission. This may not be necessary in all embodiments. Thus for example, in some embodiments it might be desirable for a laser-radiation-protective assembly to also exhibit at least some broadband blocking of visible light, whether for eye-protection reasons or simply to mitigate the annoying effects of bright sunlight. So, in some embodiments a laser-protective stack 20 as disclosed herein may comprise one or more layers that are configured to provide broadband reduction of visible light, in addition to the stack's providing narrow-band protection against laser radiation in one or more specific wavelength ranges. Such broadband attenuation of visible light may or may not necessarily be provided by a multilayer optical film. In some embodiments, a broadband visible light attenuation may take the form of a neutral-density filter of the general type described in U.S. Pat. No. 10,948,745, which is incorporated by reference herein in its entirety.

It will be apparent to those skilled in the art that the specific exemplary elements, structures, features, details, configurations, etc., that are disclosed herein can be modified and/or combined in numerous embodiments. All such variations and combinations are contemplated by the inventor as being within the bounds of the conceived invention, not merely those representative designs that were chosen to serve as exemplary illustrations. Thus, the scope of the present invention should not be limited to the specific illustrative structures described herein, but rather extends at least to the structures described by the language of the claims, and the equivalents of those structures. Any of the elements that are positively recited in this specification as alternatives may be explicitly included in the claims or excluded from the claims, in any combination as desired. Any of the elements or combinations of elements that are recited in this specification in open-ended language (e.g., comprise and derivatives thereof), are considered to additionally be recited in closed-ended language (e.g., consist and derivatives thereof) and in partially closed-ended language (e.g., consist essentially, and derivatives thereof). To the extent that there is any conflict or discrepancy between this specification and the disclosure in any document incorporated by reference herein, this specification will control.

What is claimed is:

1. A protective assembly comprising:
a singly-curved optically clear substrate comprising a front side and surface;
and,
a laser-protective stack adhesively laminated to a front side of the substrate, the laser-protective stack comprising:
a multilayer optical film configured to exhibit a light-transmission of 10% or less in a preselected wavelength range of the visible spectrum;
an optically clear physical-protection layer that is in front of the multilayer optical film and a first optically clear pressure-sensitive adhesive that adhesively bonds a rear side of the optically clear physical protection layer to a front side of the multilayer optical film;
and, a second optically clear pressure-sensitive adhesive that adhesively bonds a rear side of the multilayer optical film to a front side of the substrate;
wherein the laser-protective stack exhibits an overall light-transmission of at least 60% over the visible spectrum.

2. The protective assembly of claim 1 wherein the preselected wavelength range of the visible spectrum is within 510-550 nm.

3. The protective assembly of claim 2 wherein the preselected wavelength range that is within 510-550 nm is a first preselected wavelength range, and wherein the multilayer optical film is configured to also exhibit a light-transmission of 10% or less in a second preselected range of the visible spectrum, that is within 610-680 nm.

4. The protective assembly of claim 2 wherein the multilayer optical film that is configured to exhibit a light-transmission of less than 10% in the preselected wavelength range that is within 510-550 nm, is a first multilayer optical film and where the laser-protective stack comprises a second multilayer optical film that is configured to exhibit a light-transmission of less than 10% in a preselected range that is within 610-680 nm.

5. The protective assembly of claim 2 wherein the preselected wavelength range of the visible spectrum is within 610-680 nm.

6. The protective assembly of claim 1 wherein the laser-protective stack is configured to exhibit less than 2% transmission of infrared radiation having a wavelength of over 680 nm and is configured to exhibit less than 2% transmission of ultraviolet radiation having a wavelength of under 380 nm.

7. The protective assembly of claim 6 wherein the multilayer optical film is configured to reflectively block infrared radiation having a wavelength of over 680 nm and is configured to reflectively block ultraviolet radiation having a wavelength of under 380 nm.

8. The protective assembly of claim 6 wherein the laser-protective stack further comprises at least one additional film in addition to the multilayer optical film, the at least one additional film being configured to absorptively block the infrared radiation having a wavelength over 680 nm and/or to absorptively block the ultraviolet radiation having a wavelength of under 250 nm.

9. The protective assembly of claim 1 wherein the second optically clear adhesive comprises a thickness of from 10 microns to 100 microns.

10. The protective assembly of claim 1 wherein a front surface of the optically clear physical-protection layer comprises a scratch-resistant layer.

11. The protective assembly of claim 1 wherein a front surface of the optically clear physical-protection layer comprises an anti-reflection layer.

12. The protective assembly of claim 1 wherein the substrate exhibits an at least substantially constant radius of curvature at all locations of the substrate that lie on an arc that is centered on a midline of the substrate and that extends 45 degrees to each side of the midline along an arcuate direction of the substrate.

13. The protective assembly of claim 1 wherein the substrate is a variable thickness substrate that exhibits a maximum local thickness at a midline of the substrate and wherein the local thickness of the substrate decreases as a path is followed along an arcuate direction of the substrate away from the midline of the substrate, so that a local thickness of the substrate at a location that is 45 degrees away from the midline of the substrate along the arcuate direction of the substrate is from 60 to 80% of the local thickness of the substrate at the midline.

14. The protective assembly of claim 1 wherein the substrate is polycarbonate.

15. The protective assembly of claim 1 wherein the protective assembly is an impact-resistant pane of a protective visor or is an impact-resistant pane of protective goggles.

16. A method of making the protective assembly of claim 1, the method comprising adhesively laminating the laser-protective stack to the front side of the substrate by way of the second optically clear pressure-sensitive adhesive.

17. The method of claim 16 wherein the method is performed by passing the laser-protective stack and the substrate through a lamination nip comprising first and second rollers that are pressed toward each other.

18. The method of claim 17 wherein the substrate exhibits a local thickness that varies along an arcuate direction of the substrate, wherein the substrate is fed into the lamination nip in a direction that is aligned with the arcuate direction of the substrate, and wherein at least one of the first and second rollers comprises a resiliently deformable surface so that the roller is configured to compensate for the variable thickness of the substrate.

19. A method of protecting an eye of a user using the protective assembly of claim 1, the method comprising:
donning a headgear comprising the protective assembly of claim 1 so that the protective assembly lies in an optical path along which electromagnetic radiation travels to the eye of the user.

20. The method of claim 19 wherein the headgear comprises an impact-resistant visor or impact-resistant goggles.
